(19) United States
(12) Patent Application Publication
(10) Pub. No.: US 2018/0250235 A1
(43) Pub. Date: Sep. 6, 2018

(54) FINGOLIMOD CAPSULE COMPOSITION

(71) Applicant: SANOVEL ILAC SANAYI VE TICARET A.S., Istanbul (TR)

(72) Inventors: ALI TURKYILMAZ, Istanbul (TR); SIBEL ZENGINER, Istanbul (TR); YAVUZ DEDEOGLU, Istanbul (TR); GOKSU YAG, Istanbul (TR); ERKIN OZTURK, Istanbul (TR)

(73) Assignee: SANOVEL ILAC SANAYI VE TICARET A.S., Istanbul (TR)

(21) Appl. No.: 15/760,550

(22) PCT Filed: Sep. 16, 2016
(86) PCT No.: PCT/EP2016/071997

§ 371 Date: Mar. 15, 2018

(30) Foreign Application Priority Data
Sep. 18, 2015 (TR) 2015/11720

(51) Int. Cl. A61K 9/48; A61K 31/137

(52) U.S. Cl. CPC A61K 9/4866; A61K 9/485; A61K 31/137

(57) ABSTRACT

The present invention relates to a pharmaceutical capsule composition comprising fingolimod or its pharmaceutically acceptable salts thereof, a cellulose derivatives excipient and at least one pharmaceutically acceptable excipient.

### CROSS REFERENCE TO RELATED APPLICATIONS

This application is a § 371 National Phase Application of PCT/EP2016/071997, filed Sep. 16, 2016, which application claims priority to 2015/11720(TR), filed Sep. 18, 2015, the teachings of both of which are hereby incorporated by reference in their entireties.

### FIELD OF INVENTION

The present invention relates to a pharmaceutical capsule composition comprising fingolimod or its pharmaceutically acceptable salts thereof, cellulose derivatives excipient and at least one pharmaceutically acceptable excipient.

### BACKGROUND OF INVENTION

Multiple sclerosis (MS) is the most common autoimmune disorder affecting the central nervous system. Multiple sclerosis, also known as disseminated sclerosis or encephalomyelitis disseminata, is a demyelinating disease in which the insulating covers of nerve cells in the brain and spinal cord are damaged. This damage disrupts the ability of parts of the nervous system to communicate, resulting in a wide range of signs and symptoms, including physical, mental and sometimes psychiatric problems. MS takes several forms, with new symptoms either occurring in isolated attacks (relapsing forms) or building up over time (progressive forms). Between attacks, symptoms may disappear completely; however, permanent neurological problems often occur, especially as the disease advances.

Fingolimod is an immunomodulating drug, mostly used for treating multiple sclerosis (MS). Fingolimod is a sphingosine-1-phosphate receptor modulator, which sequesters lymphocytes in lymph nodes, preventing them from contributing to an autoimmune reaction. Fingolimod is used in the treatment of the relapsing form of multiple sclerosis. It may also be used in chronic inflammatory demyelinating polyneuropathy.

Fingolimod is marketed by Novartis under the brand name Gilenya® for the treatment of multiple sclerosis. Gilenya® is presented as immediate-release hard gelatin capsules containing 0.56 mg of fingolimod hydrochloride as the active substance corresponding to 0.5 mg of fingolimod base.

Fingolimod is 2-amino-2-[2-(4-octylphenyl)ethyl]propane-1,3-diol and shown as Formula I.

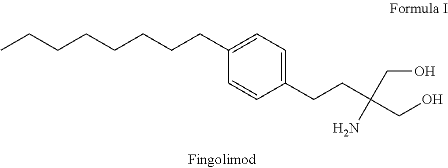 [SMILES: CCCCCCCCc1ccc(CCC(N)(CO)CO)cc1]

In prior art, fingolimod derivatives were first disclosed in U.S. Pat. No. 5,604,229. The use of fingolimod derivatives as immunodepressants and a preventive or remedy for autoimmune diseases is disclosed in EP0627406 (B1). The use of fingolimod derivatives in the prevention or treatment of chronic rejection in a recipient of organ or tissue allo- or xenotransplant is disclosed in EP0941082 (B1).

WO201319872 addresses the problem of stability by mixing fingolimod with a dry surfactant and then formulating the resulting pre-mix.

1844/CHE/2011 discloses a solid oral composition comprising fingolimod, pregelatinized starch and stearic acid, but the dissolution profile of this composition is slower when compared with Gilenya® product. Moreover, the patent application does not provide any stability study to show compatibility of excipients with fingolimod.

It has been discovered that fingolimod possesses properties that can cause several processing problems like content uniformity as fingolimod particles have a strong tendency to stick to surfaces and each other.

Despite various prior art availability to provide a solution of one or the other issues associated with fingolimod hydrochloride, still there exists a need for selecting excipients for formulating fingolimod, which provides a stable product and provides desired therapeutic effect.

The present invention overcomes all the above mentioned problems by providing a stable solid oral capsule composition having desired content uniformity and dissolution profile. It can be dispensed as an oral dosage form to overcome the several processing problems associated with the fingolimod composition.

### DESCRIPTION OF THE INVENTION

This invention is about a pharmaceutical capsule composition comprising fingolimod or its pharmaceutically acceptable salts thereof, a cellulose derivatives excipient and at least one pharmaceutically acceptable excipient.

In a preferred embodiment, a pharmaceutically acceptable salt of fingolimod is fingolimod hydrochloride.

In the marketed fingolimod product (Gilenya®), the capsule comprises mannitol as a diluent, prepared by a direct blending method and capsule, which additionally comprises a small amount of magnesium stearate as a lubricant. Fingolimod HCl was used in a micronized form to ensure content uniformity of the active substance. It is disclosed in the literature that fingolimod reacts with several excipients during compatibility studies and thus, mannitol was used as a diluent in the Gilenya® composition due to the optimum degradation profile. Since the dose of the approved product is very low, and filler or diluent makes most of the part of the composition it is essential to select an excipient which is highly compatible with API. (European public assessment report of Gilenya®)

Both in the marketed fingolimod product and in this present invention, the hard gelatin capsules containing 0.56 mg of fingolimod hydrochloride as the active substance correspond to 0.5 mg of fingolimod base. The proportion of the active agent in the total weight of the composition in the case of compositions for oral administration is typically in the range of only a few percent by weight, such as 0.25 to 4% by weight. This small proportion of active agent can lead to considerable problems during the manufacture of the composition with regard to the uniformity of the content of active agent in the individual composition units. The compatibility studies showed that the active substance interacts with several excipients. It reflects that content uniformity plays an important role in the dissolution of the drug. According to this embodiment, the amount of fingolimod hydrochloride is present in an amount of 0.25 to 4% by weight of total composition; preferably it is 0.5 to 2.5% by weight of total composition.

The present invention is based on the findings that pharmaceutical compositions and dosage forms comprising fingolimod hydrochloride having improved content uniformity and/or flowability and/or dissolution is achieved by using cellulose and/or its derivatives, which are chosen based on compatibility studies and experience from development of similar compositions.

In this present invention, microcrystalline cellulose PH 101 is used in in this present capsule composition to improve flow, facilitate plug formation and aid capsule disintegration.

According to a preferred embodiment, the pharmaceutical capsule composition comprising a cellulose derivatives excipient is microcrystalline cellulose PH 101.

In one embodiment, the amount of microcrystalline cellulose PH 101 is present in an amount of 35.0 to 75.0% by weight of total composition; preferably it is 45.0 to 65.0% by weight of total composition.

An object of the present invention is to obtain adequate content uniformity and desired particle size distribution of composition comprising fingolimod hydrochloride and microcrystalline cellulose PH 101 and improved processes. In this invention, the composition has homogeneity and high flowability properties during the process.

As used herein, “particle size distribution” is defined by the cumulative volume size distribution as tested by a conventionally accepted method which is the laser diffraction method determined by the equipment of Malvern Mastersizer 2000. “Median particle size” is defined by “d50”, the diameter where fifty percent of the distribution has a smaller particle size and “d90” means that the diameter where ninety percent of the distribution has a smaller particle size. “Desired particle size distribution” is defined by the ratio between the median particle size (d50) and the particle size at (d90).

In one embodiment, the pharmaceutical capsule composition comprises microcrystalline cellulose PH 101 having a particle size (d₅₀) between 50.0 μm and 95.0 μm, and preferably between 60.0 μm and 85.0 μm.

In another embodiment, the pharmaceutical capsule composition comprises microcrystalline cellulose PH 101 having a particle size (d₉₀) between 125.0 μm and 165.0 μm, and preferably between 135.0 μm and 155.0 μm.

According to the special particle size of microcrystalline cellulose PH 101 in the composition, the invention's advantages are a reasonable preparing process, high flowability, uniform dispersion and desired dissolution profile. It has been found that this specific particle size improves content uniformity and flowability. In this present invention, the composition can be easily handled, mixed with the excipients having a particular particle size and can be processed into a pharmaceutical product without the known and expected problems such as segregation or de-mixing during the process steps.

According to one embodiment, the pharmaceutical capsule composition comprises at least one pharmaceutically acceptable excipient which is selected from diluent, binder, lubricant or mixtures thereof.

Suitable diluents may comprise, but are not limited to, microcrystalline cellulose, microcrystalline cellulose PH 101, calcium hydrogen phosphate dihydrate, lactose spray dried, mannitol, spray-dried mannitol, lactose, starch, maize starch, dextrose, sucrose, fructose, maltose, sorbitol, xylitol, inositol, kaolin, inorganic salts, calcium salts, polysaccharides, calcium phosphate, dicalcium phosphate, sodium chloride, dextrates, lactitol, maltodextrin, sucrose-maltodextrin mixture, trehalose, sodium carbonate, sodium bicarbonate, calcium carbonate or mixtures thereof.

According to another preferred embodiment, the pharmaceutical capsule composition comprising a diluent is calcium hydrogen phosphate dihydrate.

In one embodiment, the amount of calcium hydrogen phosphate dihydrate is present in an amount of 20.0 to 60.0% by weight of the total composition; preferably it is 30.0 to 50.0% by weight of the total composition.

Suitable binders may include, but are not limited to, polyethylene glycol, polyethylene glycol 6000 P, polyvinylpyrrolidone, polyvinyl alcohol, starch, pregelatinized starch, glucose, glucose syrup, natural gums, sucrose, sodium alginate, hydroxypropyl methyl cellulose, hydroxypropyl cellulose, carboxy methyl cellulose, methyl cellulose, gelatin, carrageenan, guar gum, carbomer, polymethacrylates, methacrylate polymers, collagens gelatin, agar, alginate, alginic acid, xanthan gum, hyaluronic acid, pectin, polysaccharides, carbomer, poloxamer, polyacrylamide, aluminium hydroxide, laponit, bentonit, polyoxyethylene-alkyl ether, polydextrose, polyethylene oxide or mixtures thereof.

In this present invention, polyethylene glycol 6000 P is selected as a suitable binder. Polyethylene glycol 6000 P is a polyethylene glycol with a mean molecular weight of 6000. It is a solid in powder form. It has very low water content and excellent solubility in water. In this present invention, polyethylene glycol 6000 P is used in the wet granulation process, the risk of over-granulation is reduced, screen blockage is avoided and uniform, rapid drying is achieved.

In one embodiment, the amount of polyethylene glycol 6000 P is present in an amount of 0.5 to 5.0% by weight of the total composition; preferably it is 1.0 to 4.0% by weight of the total composition.

Suitable lubricants may comprise, but are not limited to, sodium stearyl fumarate, magnesium stearate, calcium stearate, zinc stearate, waxes, boric acid, hydrogenated vegetable oil, sodium chlorate, magnesium lauryl sulfate, sodium oleate, sodium acetate, sodium benzoate, stearic acid, fatty acid, fumaric acid, glyceryl palmito sulphate, sodium lauryl sulphate or mixtures thereof.

In this present invention, sodium stearyl fumarate is selected as a suitable lubricant. Due to the hydrophilic property of sodium stearyl fumarate, the composition does not have the disadvantages of magnesium stearate in respect of flowability and dissolution. In one aspect, the present invention relates to a fingolimod hydrochloride capsule composition which does not comprise magnesium stearate. Magnesium stearate is a metal derivative lubricant. It has anti-adhesive and flow enhancement properties by ensuring uniformity. Besides this advantage, the lubricant forms a hydrophobic film around the active agent and retards the dissolution by retarding wetting of the active agent. In this invention, sodium stearyl fumarate helps to achieve the desired dissolution profile.

In one embodiment, the amount of sodium stearyl fumarate is present in an amount of 0.5 to 5.0% by weight of the total composition; preferably it is 1.0 to 4.0% by weight of the total composition.

In one embodiment, the pharmaceutical capsule composition consisting of;
a. fingolimod hydrochloride is present in an amount of 0.25 to 4% by weight of total composition; preferably it is 0.5 to 2.5% by weight of total composition;
b. microcrystalline cellulose PH 101 is present in an amount of 35.0 to 75.0% by weight of total composition; preferably it is 45.0 to 65.0% by weight of total composition;
c. calcium hydrogen phosphate dihydrate is present in an amount of 20.0 to 60.0% by weight of total composition; preferably it is 30.0 to 50.0% by weight of total composition;
d. polyethylene glycol 6000 P is present in an amount of 0.5 to 5.0% by weight of total composition; preferably it is 1.0 to 4.0% by weight of total composition; and
e. sodium stearyl fumarate is present in an amount of 0.5 to 5.0% by weight of total composition; preferably it is 1.0 to 4.0% by weight of total composition.

In a preferred embodiment, the pharmaceutical capsule composition comprises;
a. fingolimod hydrochloride;
b. microcrystalline cellulose PH 101 having a particle size (d₅₀) between 50.0 μm and 95.0 μm, and preferably between 60.0 μm and 85.0 μm;
c. calcium hydrogen phosphate dehydrate;
d. polyethylene glycol 6000 P; and
e. sodium stearyl fumarate.

Another object of the present invention is to provide a pharmaceutical capsule composition comprising fingolimod or its pharmaceutically acceptable salt thereof for the treatment of multiple sclerosis, preferably relapsing-remitting multiple sclerosis.

### Example: Fingolimod Hydrochloride Capsule Composition

Microcrystalline cellulose PH 101: d₅₀=50.0 μm-95.0 μm; d₉₀=125.0 μm-165.0 μm

| Ingredients | Amount (mg) |
|---|---|
| Fingolimod hydrochloride | 0.56 |
| Microcrystalline cellulose PH 101 | 27.44 |
| Calcium hydrogen phosphate dihydrate | 20.00 |
| Polyethylene glycol 6000 P | 1.00 |
| Sodium stearyl fumarate | 1.00 |

Manufacturing Process: Fingolimod hydrochloride, microcrystalline cellulose PH 101, calcium hydrogen phosphate dihydrate and polyethylene glycol 6000 P is blended progressively. The mixture is granulated with ethanol:water (1:1) solution. The granules were dried at 35° C. and sieved. Sodium stearyl fumarate is added to the final mixture. Then the powder is filled to the hard gelatin capsules.

1. A pharmaceutical capsule composition comprising fingolimod or its pharmaceutically acceptable salts thereof, a cellulose derivatives excipient and at least one pharmaceutically acceptable excipient.

2. The pharmaceutical capsule composition according to claim 1, wherein the pharmaceutically acceptable salt of fingolimod is fingolimod hydrochloride.

3. The pharmaceutical capsule composition according to claim 1, wherein the cellulose derivatives excipient is microcrystalline cellulose PH 101.

4. The pharmaceutical capsule composition according to claim 3, wherein microcrystalline cellulose PH 101 is present in an amount of 35.0 to 75.0% by weight of total composition.

5. The pharmaceutical capsule composition according to claim 3, wherein microcrystalline cellulose PH 101 has a particle size (d₅₀) between 50.0 μm and 95.0 μm.

6. The pharmaceutical capsule composition according to claim 1, wherein at least one pharmaceutically acceptable excipient is selected from a diluent, a binder, a lubricant or mixtures thereof.

7. The pharmaceutical capsule composition according to claim 6, wherein the diluent is calcium hydrogen phosphate dihydrate.

8. The pharmaceutical capsule composition according to claim 6, wherein the binder is polyethylene glycol 6000 P.

9. The pharmaceutical capsule composition according to claim 6, wherein the lubricant is sodium stearyl fumarate.

10. The pharmaceutical capsule composition according to claim 1, comprising;
a. fingolimod hydrochloride;
b. microcrystalline cellulose PH 101 having a particle size (d50) between 50.0 μm and 95.0 μm;
c. calcium hydrogen phosphate dehydrate;
d. polyethylene glycol 6000 P; and
e. sodium stearyl fumarate.
